(19) United States
(12) Patent Application Publication
(10) Pub. No.: US 2020/0383732 A1
(43) Pub. Date: Dec. 10, 2020

(54) POWERLESS ELECTROMAGNETIC SENSOR AND SURGICAL NAVIGATION SYSTEM INCLUDING SAME

(71) Applicants: KOREA ADVANCED INSTITUTE OF SCIENCE AND TECHNOLOGY, Daejeon (KR); KYUNGPOOK NATIONAL UNIVERSITY INDUSTRY-ACADEMIC COOPERATION FOUNDATION, Daegu (KR)

(72) Inventors: Min Kyu JE, Daegu (KR); Jun Young KIM, Daegu (KR); Cheol Jun PARK, Daejeon (KR); Jae Suk CHOI, Daejeon (KR); Hong Seok SHIN, Daejeon (KR); Shin Yoon KIM, Daegu (KR)

(73) Assignees: KOREA ADVANCED INSTITUTE OF SCIENCE AND TECHNOLOGY, Daejeon (KR); KYUNGPOOK NATIONAL UNIVERSITY INDUSTRY-ACADEMIC COOPERATION FOUNDATION, Daegu (KR)

(21) Appl. No.: 16/769,488

(22) PCT Filed: Dec. 12, 2018
(86) PCT No.: PCT/KR2018/015731

§ 371 Date: Jun. 3, 2020

(30) Foreign Application Priority Data
Dec. 12, 2017 (KR) 10-2017-0170670

(51) Int. Cl. A61B 34/20; H02J 50/12; H02J 50/80

(52) U.S. Cl. CPC A61B 34/20; A61B 2034/2051; H02J 50/80; H02J 50/12

(57) ABSTRACT

A powerless electromagnetic sensor according to an embodiment may comprise: a wireless power receiver for receiving a power signal from an external data receiver unit by using one resonance frequency selected from a plurality of resonance frequencies set therefor; a digital signal converter for converting the received power signal into a digital signal; and a processor for converting the power signal into a magnetic flux density value corresponding to the selected resonance frequency on the basis of the digital signal, and calculating the position and orientation of the electromagnetic sensor on the basis of the converted magnetic flux density value.

### TECHNICAL FIELD

Hereinafter, embodiments relate to a powerless electromagnetic sensor and a surgical navigation system including the same.

### BACKGROUND ART

An electromagnetic sensor detects information derived from an electromagnetic field generated by an electromagnetic wave generator to calculate the position and orientation of an object in which the electromagnetic sensor is provided. In a conventional electromagnetic sensor, a sensor interface connected to a processor (e.g. computer) that performs a series of signal processing is “connected through wires” to transmit data. The electromagnetic sensor using this scheme requires the wires connecting the processor to the electromagnetic sensor provided in the object even if it is in small size, and thus, there is a limitation in the range of application of the electromagnetic sensor. Moreover, in case of using a plurality of electromagnetic sensors, the wires, of which the number is proportional to the number of the electromagnetic sensors being used, are essential.

Various solutions to address these limitations have been proposed. In an example, there is a method in which a processor calculates the position and orientation of an object in which an optical sensor is provided by measuring light emitted/reflected from the optical sensor through a camera (also referred to as an optical tracking method). This method should secure a path of light between the camera and the optical sensor. If another object placed between the optical sensor and the camera blocks the path of light, this method may not be used. In addition, the optical sensor used in this method is greater than the electromagnetic sensor, and thus there is a limitation in the range of application of the optical sensor. For example, Korean Patent Application Publication No. 10-2003-0082942 discloses total knee arthroplasty systems and processes.

In another example, there is a sensor using a wireless interface with a built-in battery. This sensor wirelessly communicates with an external processor (e.g. computer) through the wireless interface. However, this sensor has the wireless interface including the battery therein, and thus the sensor and its accessory(s) attached to an object to be detected are in large size.

### DISCLOSURE OF INVENTION

### Technical Goals

An aspect provides a powerless electromagnetic sensor that is energy-autonomous and ultra-compact using wireless power harvesting, and a surgical navigation system including the same.

### Technical Solutions

According to an aspect, there is provided a powerless electromagnetic sensor including a wireless power receiver configured to receive a power signal from an external data receiver unit by using a resonance frequency selected from a plurality of resonance frequencies set therefor, a digital signal converter configured to convert the received power signal into a digital signal, and a processor configured to convert the power signal into a magnetic flux density value corresponding to the selected resonance frequency on the basis of the digital signal, and calculate the position and orientation of the electromagnetic sensor on the basis of the converted magnetic flux density value.

The wireless power receiver may be configured to sequentially select the plurality of set resonance frequencies and sequentially receive power signals corresponding to the selected resonance frequencies from the external data receiver unit.

The processor may be configured to calculate the position and orientation of the electromagnetic sensor on the basis of magnetic flux density values respectively corresponding to the plurality of set resonance frequencies.

The wireless power receiver may be configured to convert the power signal of the selected resonance frequency from the alternating current (AC) form to the direct current (DC) form.

The wireless power receiver may include a coil configured to pass the power signal from the external data receiver unit therethrough, and a plurality of capacitors to be coupled with the coil to have resonance frequencies respectively corresponding to the plurality of resonance frequencies, wherein the coil may be selectively coupled with one of the plurality of capacitors.

The powerless electromagnetic sensor may further include a power management unit configured to receive the converted power signal and manage the power of the digital signal converter and the processor.

The powerless electromagnetic sensor may further include a wireless communication unit configured to wirelessly transmit information related to the position and orientation of the electromagnetic sensor calculated by the processor to the external data receiver unit.

According to an aspect, there is provided a surgical navigation system including an electromagnetic wave generator configured to generate an electromagnetic wave, an electromagnetic sensor provided in a patient-specific instrument (PSI), and configured to receive a power signal from the electromagnetic wave generator by using a resonance frequency selected from a plurality of resonance frequencies set therefor, and a processor configured to convert the received power signal into a digital signal, convert the power signal into a magnetic flux density value corresponding to the selected resonance frequency on the basis of the digital signal, calculate a position and orientation of the electromagnetic sensor on the basis of the converted magnetic flux density value, and calculate a position and orientation of the PSI on the basis of the position and orientation of the electromagnetic sensor.

### Effects

According to embodiments, a powerless electromagnetic sensor and a surgical navigation system including the same may have no limitation in the range of application to an object.

According to embodiments, a powerless electromagnetic sensor and a surgical navigation system including the same may miniaturize a sensor provided in an object.

The effects of the powerless electromagnetic sensor and the surgical navigation system including the same are not limited to the above-mentioned effects. And, other unmentioned effects can be clearly understood from the above description by those having ordinary skill in the technical field to which the present disclosure pertains.

### BRIEF DESCRIPTION OF DRAWINGS

FIG. 1 is a block diagram illustrating a powerless electromagnetic sensor according to an embodiment.

FIG. 2 is a circuit diagram illustrating a wireless power receiver of a powerless electromagnetic sensor according to an embodiment.

FIG. 3 is a flowchart illustrating a method of processing a wireless power signal by a powerless electromagnetic sensor according to an embodiment.

FIG. 4 illustrates signal flows according to the position of the powerless electromagnetic sensor of FIG. 1.

FIG. 5 is a perspective view illustrating a surgical navigation system to which a powerless electromagnetic sensor is applied according to an embodiment.

### BEST MODE FOR CARRYING OUT THE INVENTION

Hereinafter, embodiments will be described in detail with reference to the illustrative drawings. Regarding the reference numerals assigned to the components in the drawings, it should be noted that the same components will be designated by the same reference numerals, wherever possible, even though they are shown in different drawings. Further, in the following description of the present embodiments, a detailed description of publicly known configurations or functions incorporated herein will be omitted when it is determined that the detailed description obscures the subject matters of the present embodiments.

In addition, the terms first, second, A, B, (a), and (b) may be used to describe constituent elements of the embodiments. These terms are used only for the purpose of discriminating one constituent element from another constituent element, and the nature, the sequences, or the orders of the constituent elements are not limited by the terms. When one constituent element is described as being “connected”, “coupled”, or “attached” to another constituent element, it should be understood that one constituent element can be connected or attached directly to another constituent element, and an intervening constituent element can also be “connected”, “coupled”, or “attached” to the constituent elements.

The constituent element, which has the same common function as the constituent element included in any one embodiment, will be described by using the same name in other embodiments. Unless disclosed to the contrary, the configuration disclosed in any one embodiment may be applied to other embodiments, and the specific description of the repeated configuration will be omitted.

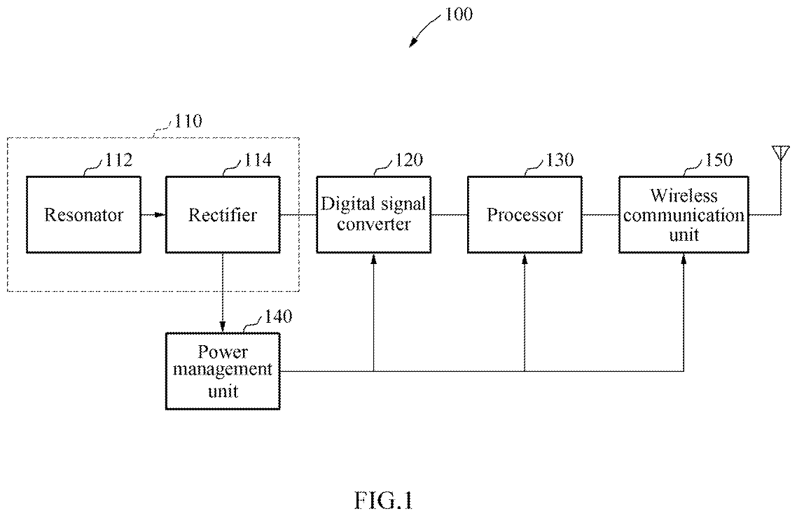

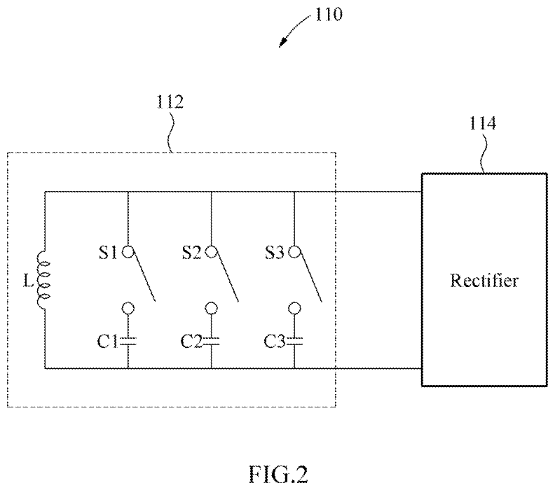

FIG. 1 is a block diagram illustrating a powerless electromagnetic sensor according to an embodiment, and FIG. 2 is a circuit diagram illustrating a wireless power receiver of the powerless electromagnetic sensor according to an embodiment.

A powerless electromagnetic sensor 100 may not be provided with an element (e.g. a battery, and the like) that supply power to the electromagnetic sensor 100, and may operate by wirelessly receiving a power signal from an external data receiver unit. The powerless electromagnetic sensor 100 may include a wireless power receiver 110, a digital signal converter 120, a processor 130, a power management unit 140, and a wireless communication unit 150.

The wireless power receiver 110 may receive the power signal from the external data receiver unit. Here, the power signal may be in the form of a magnetic field or electromagnetic field having a characteristic of oscillation. The wireless power receiver 110 may include a resonator 112 and a rectifier 114.

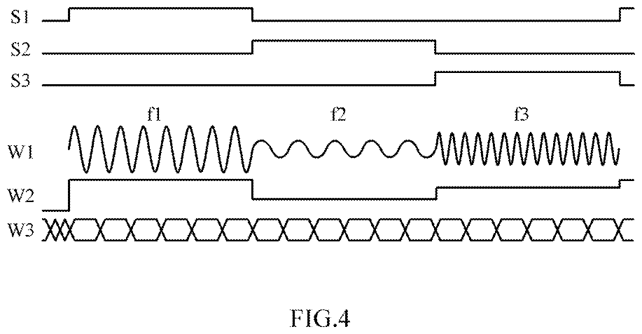

The resonator 112 may receive a power signal having a set resonance frequency from the external data receiver unit in a resonance coupling manner. The resonator 112 may include a coil L through which the power signal (e.g. the electromagnetic field) passes and a plurality of capacitors. Here, the number of capacitors is not limited. However, for convenience of description, three capacitors C1, C2, and C3 will be described herein. The coil L and the plurality of capacitors C1, C2, and C3 may be connected in parallel. According to the inductance of the coil L and the capacitance of each of the plurality of capacitors C1, C2, and C3, a plurality of resonance frequencies f1, f2, and f3 (see FIG. 4) may be set.

The resonator 112 may select one of the plurality of set resonance frequencies. Here, the number of resonance frequencies is not limited. However, for convenience of description, three resonance frequencies f1, f2, and f3 (see FIG. 4) will be described herein. The resonator 112 may include a plurality of switches. Here, the number of switches is not limited. However, for convenience of description, three switches S1, S2, and S3 will be described herein. The plurality of switches S1, S2, and S3 may be selectively opened or closed. In order for the resonator 112 to receive a power signal of the first resonance frequency f1, the first switch S1 may be closed and the second switch S2 and the third switch S3 may be opened, such that the coil L and the first capacitor C1 may be coupled. Likewise, in order for the resonator 112 to receive a power signal of the second resonance frequency f2, the second switch S2 may be closed and the first switch S1 and the third switch S3 may be opened, such that the coil L and the second capacitor C2 may be coupled. Likewise, in order for the resonator 112 to receive a power signal of the third resonance frequency f3, the third switch S3 may be closed and the first switch S1 and the second switch S2 may be opened, such that the coil L and the third capacitor C3 may be coupled. Consequently, as the first switch S1, the second switch S2, and/or the third switch S3 are selectively opened or closed, a power signal of one of the first resonance frequency f1, the second resonance frequency f2, and the third resonance frequency f3 may be received.

In an embodiment, the resonator 112 may sequentially select the plurality of set resonance frequencies f1, f2, and f3 (see FIG. 4) and sequentially receive power signals corresponding to the selected resonance frequencies from the external data receiver unit. For example, the resonance frequencies are selected in the order of the first resonance frequency f1, the second resonance frequency f2, and the third resonance frequency f3. However, the order is not limited thereto.

In an embodiment, the resonator 112 may not receive a power signal by selecting another resonance frequency immediately after receiving a power signal by selecting one resonance frequency, but may rather receive a power signal by selecting another resonance frequency after a power signal received previously goes through a series of signal processing process.

The rectifier 114 may convert an alternating current (AC) power signal of the set resonance frequency received from the resonator 112 into a direct current (DC) power signal.

Although not shown, a direct current-to-direct current (DC/DC) converter may be additionally provided between the rectifier 114 and the digital signal converter 120 or between the rectifier 114 and the power management unit 140. The DC/DC converter outputs a rated voltage or current by adjusting the level of the DC power signal converted by the rectifier 114.

The digital signal converter 120 may convert the power signal received from the wireless power receiver 110, that is, the DC power signal converted by the rectifier 114, into a digital signal. Since the power signal of the set resonance frequency selectively received from the resonator 112 is converted into a digital signal, the energy efficiency when processing the digital signal is higher than the energy efficiency when processing the analog signal.

The processor 130 may convert the power signal into a magnetic flux density value on the basis of the digital signal converted by the digital signal converter 120. Here, the converted magnetic flux density value corresponds to the magnitude of the power signal received at the resonance frequency at the time of selection. The processor 130 may calculate the position and orientation of the electromagnetic sensor 100 on the basis of the converted magnetic flux density value.

In an embodiment, when the resonator 112 sequentially selects the plurality of resonance frequencies, the processor 130 may convert a power signal of a resonance frequency selected first by the resonator 112 into a magnetic flux density value, and the resonator 112 may select one of the remaining resonance frequencies that are not selected from among the plurality of resonance frequencies and receive a power signal corresponding thereto. In other words, the resonator 112 may receive a power signal by selecting a remaining resonance frequency other than a previous resonance frequency from among the plurality of resonance frequencies, based on a time point at which the processor 130 converts the power signal into a magnetic flux density value.

In an embodiment, the processor 130 may sequentially convert the power signals respectively corresponding to the plurality of resonance frequencies, from the resonator 112, into magnetic flux density values, and then calculate the position and orientation of the electromagnetic sensor 100 on the basis of the plurality of magnetic flux density values.

The power management unit 140 may receive the power signal converted by the wireless power receiver 110 and manage the power of the digital signal converter 120, the processor 130, and the wireless communication unit 150. For example, the power management unit 140 may be a power management integrated circuit (PMIC), a power charging integrated circuit (IC), or the like. Since the power management unit 140 manages the power of the electromagnetic sensor 100 on the basis of the power signal received from the external data receiver unit, a large capacity of a battery is not required.

The wireless communication unit 150 may wirelessly transmit information regarding the position and orientation of the electromagnetic sensor 100 calculated by the processor 130 to the external data receiver unit. For example, the wireless communication unit 150 may use wireless fidelity (WiFi), Bluetooth, near field communication (NFC).

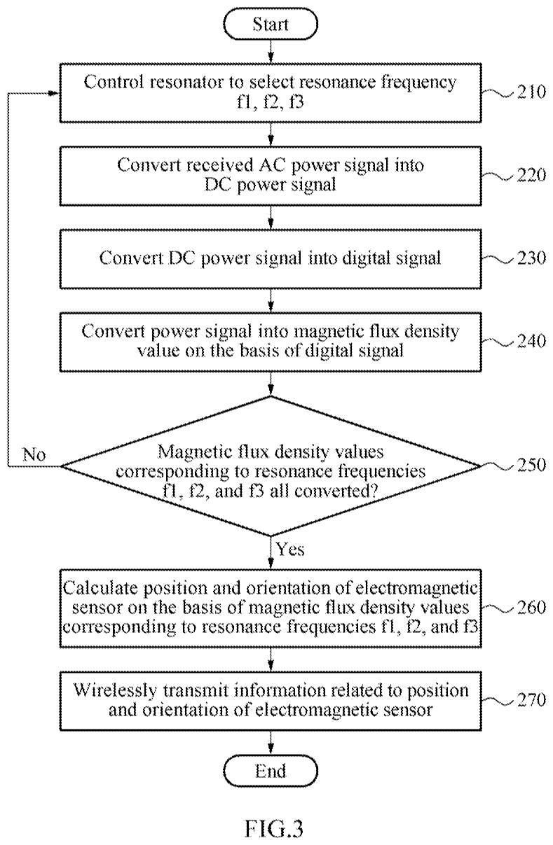

FIG. 3 is a flowchart illustrating a method of processing a wireless power signal by a powerless electromagnetic sensor according to an embodiment.

Referring to FIG. 3, a method of processing a wireless power signal by a powerless electromagnetic sensor first controls a resonator to select one of a plurality of resonance frequencies f1, f2, and f3 and receives a power signal, in operation 210. Thereafter, the method converts the AC power signal of the selected resonance frequency into a DC power signal, in operation 220. Thereafter, the method converts the DC power signal into a digital signal, in operation 230. Thereafter, the method converts the received power signal into a magnetic flux density value corresponding to the selected resonance frequency on the basis of the converted digital signal in operation 240.

Thereafter, the method checks whether the magnetic flux density values respectively corresponding to the plurality of resonance frequencies f1, f2, and f3 are all generated, in operation 250. If the magnetic flux density values respectively corresponding to the plurality of resonance frequencies f1, f2, and f3 are not all generated, the method returns again to operation 210 of receiving a power by selecting remaining resonance frequency(s) that are not selected, from among the plurality of resonance frequencies, and repeats operations 220 to 240. If the magnetic flux density values respectively corresponding to the plurality of resonance frequencies f1, f2, and f3 are all generated, the method calculates the position and orientation of the electromagnetic sensor on the basis of the magnetic flux density values respectively corresponding to the plurality of resonance frequencies f1, f2, and f3, in operation 260.

Thereafter, the method may wirelessly transmit information related to the calculated position and orientation of the electromagnetic sensor to an external data receiver unit, in operation 270.

FIG. 4 illustrates signal flows according to the position of the powerless electromagnetic sensor of FIG. 1.

Referring to FIGS. 1 and 4 together, S1, S2, and S3 in FIG. 4 show closing or opening of the first switch S1, the second switch S2, and the third switch S3 according to the time in FIG. 1. In addition, W1, W2, and W3 in FIG. 4 show power signals of one of the plurality of resonance frequencies f1, f2, and f3 received according to the time in response to selective closing or opening of the first switch S1, the second switch S2, and the third switch S3 of FIG. 1. Here, W1 is a signal between the resonator 112 and the rectifier 114 of FIG. 1, W2 is a signal between the rectifier 114 and the digital signal converter 120 of FIG. 1, and W3 is a signal between the digital signal converter 120 and the processor 130 of FIG. 1.

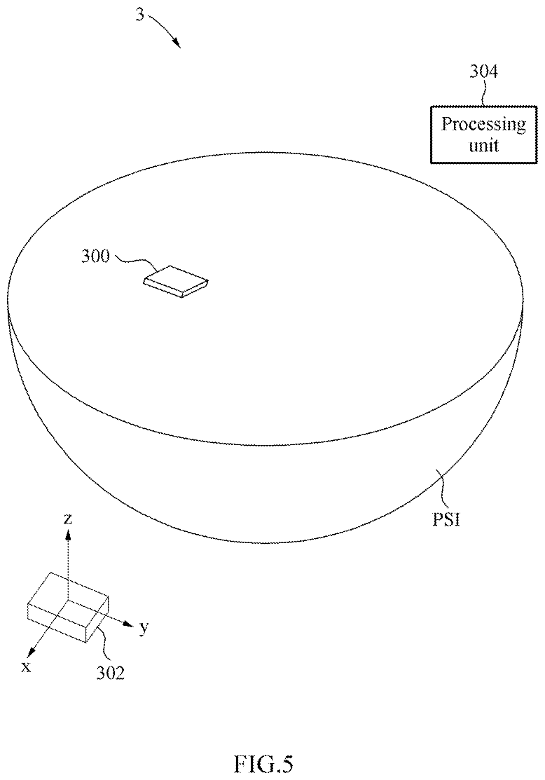

FIG. 5 is a perspective view illustrating a surgical navigation system to which a powerless electromagnetic sensor is applied according to an embodiment.

Referring to FIG. 5, a surgical navigation system 3 may include an electromagnetic sensor 300 provided in a patient-specific instrument (PSI), an electromagnetic wave generator 302, and a processing unit 304. Here, the PSI refers to an instrument that is inserted into an affected part of a human body (e.g. acetabulum) during a surgical operation (e.g. total joint replacement), and is used to register an absolute position of the affected part of the human body for setting the axis of another surgical instrument (e.g. a reamer) to be inserted into the affected part of the patient.

The electromagnetic sensor 300 may be provided in the (PSI) to calculate the position and orientation of the PSI on the basis of an electromagnetic field generated by the electromagnetic wave generator 302 and wirelessly transmit information related to the calculated position and orientation of the PSI to the processing unit 304. The structure and function of the electromagnetic sensor 300 may be clearly understood by a person skilled in the art from the description of the electromagnetic sensor provided above with reference to FIGS. 1 to 4.

The electromagnetic wave generator 302 may generate an electromagnetic field. Unlike the electromagnetic sensor 300, the electromagnetic wave generator 302 may be disposed outside of the PSI.

The processing unit 304 may receive the information related to the calculated position and orientation of the PSI from the electromagnetic sensor 300 and register the coordinates corresponding to the position of the PSI.

In an embodiment, the processing unit 304 may perform the processing scheme performed by the electromagnetic sensor 300. That is, the processing unit 304 may generate a digital signal on the basis of a power signal received from the electromagnetic sensor 300, convert the power signal into a magnetic flux density value corresponding to a selected set resonance frequency, and calculate the position and orientation of the PSI, in which the electromagnetic sensor 300 is provided, on the basis of the converted magnetic flux density value. In this example, a digital signal conversion circuit or a signal processing circuit may be omitted from the electromagnetic sensor 300.

The methods according to the above-described examples may be recorded in non-transitory computer-readable media including program instructions to implement various operations of the above-described examples. The media may also include, alone or in combination with the program instructions, data files, data structures, and the like. The program instructions recorded on the media may be those specially designed and constructed for the purposes of examples, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher-level code that may be executed by the computer using an interpreter. The above-described devices may be configured to act as one or more software modules in order to perform the operations of the above-described examples, or vice versa.

A number of embodiments have been described above. Nevertheless, it should be understood that various modifications may be made to these embodiments. For example, suitable results may be achieved if the described techniques are performed in a different order and/or if components in a described system, architecture, device, or circuit are combined in a different manner and/or replaced or supplemented by other components or their equivalents.

1. A powerless electromagnetic sensor, comprising:
a wireless power receiver configured to receive a power signal from an external data receiver unit by using a resonance frequency selected from a plurality of resonance frequencies set therefor;
a digital signal converter configured to convert the received power signal into a digital signal; and
a processor configured to convert the power signal into a magnetic flux density value corresponding to the selected resonance frequency on the basis of the digital signal, and calculate the position and orientation of the electromagnetic sensor on the basis of the converted magnetic flux density value.

2. The powerless electromagnetic sensor of claim 1, wherein the wireless power receiver is configured to sequentially select the plurality of set resonance frequencies and sequentially receive power signals corresponding to the selected resonance frequencies from the external data receiver unit.

3. The powerless electromagnetic sensor of claim 2 wherein the processor is configured to calculate the position and orientation of the electromagnetic sensor on the basis of magnetic flux density values respectively corresponding to the plurality of set resonance frequencies.

4. The powerless electromagnetic sensor of claim 1, wherein the wireless power receiver is configured to convert the power signal of the selected resonance frequency from the alternating current (AC) form to the direct current (DC) form.

5. The powerless electromagnetic sensor of claim 1, wherein the wireless power receiver comprises:
a coil configured to pass the power signal from the external data receiver unit therethrough; and
a plurality of capacitors to be coupled with the coil to have resonance frequencies respectively corresponding to the plurality of resonance frequencies,
wherein the coil is selectively coupled with one of the plurality of capacitors.

6. The powerless electromagnetic sensor of claim 1, further comprising:
a power management unit configured to receive the converted power signal and manage the power of the digital signal converter and the processor.

7. The powerless electromagnetic sensor of claim 1, further comprising:
a wireless communication unit configured to wirelessly transmit information related to the position and orientation of the electromagnetic sensor calculated by the processor to the external data receiver unit.

8. A surgical navigation system, comprising:
an electromagnetic wave generator configured to generate an electromagnetic wave;
an electromagnetic sensor provided in a patient-specific instrument (PSI), and configured to receive a power signal from the electromagnetic wave generator by using a resonance frequency selected from a plurality of resonance frequencies set therefor; and
a processor configured to convert the received power signal into a digital signal, convert the power signal into a magnetic flux density value corresponding to the selected resonance frequency on the basis of the digital signal, calculate a position and orientation of the electromagnetic sensor on the basis of the converted magnetic flux density value, and calculate a position and orientation of the PSI on the basis of the position and orientation of the electromagnetic sensor.
